(19) United States
(12) Patent Application Publication
(10) Pub. No.: US 2022/0390131 A1
(43) Pub. Date: Dec. 8, 2022

(54) TECHNIQUES FOR DISINFECTION AND DEODORIZATION OF INTERIOR OF BUILDINGS USING OZONE

(71) Applicant: Clear Path Holdings Corp., Morganville, NJ (US)

(72) Inventor: ANDREJS SAPONENKO, Dubai (AE)

(73) Assignee: Clear Path Holdings Corp., Morganville, NJ (US)

(21) Appl. No.: 17/339,705

(22) Filed: Jun. 4, 2021

(51) Int. Cl. F24F 8/40; F24F 11/38; F24F 11/58

(52) U.S. Cl. CPC F24F 8/40; F24F 11/38; F24F 11/58; F24F 2110/10

(57) ABSTRACT

The present invention solves the problem of efficiently disinfecting and deodorizing indoor areas with ozone, without introducing health risks to people inside these areas. A cold plasma ozone generator generates ozone that is distributed to one or more rooms where disinfection and/or deodorization is needed. The distribution of ozone is regulated by valves controlled by a main control module collecting data from ozone concentration and IR motion detectors installed at rooms where the ozone is distributed, and other sensors in the ozone generator, and by using a schedule associating information derived from the sensory data, with dates, time slots, and desired ozone concentrations. If ozone concentrations exceed predefined levels, ozone generation and/or distribution is interrupted. When malfunctions occur, either the main control module commands the system to enter in emergency shutdown or lockdown, or a local control module or the ozone generator take control of the ozone generation and distribution.

### BACKGROUND

### Field

The present invention relates to techniques for disinfection and deodorization of interior of buildings using ozone, and in particular to methods, systems and software for controlling the supply of ozone to indoor areas for disinfection and deodorization purposes.

### Background Information

Ozone has been known for years as a very strong oxidation agent. It has been used over the years for disinfection and deodorization by exploiting its oxidation efficacy which has proved to be particularly effective in destroying micro-organisms, such as pathogens posing a health hazard, and bacteria creating bad odors. Among ozone's applications are disinfection of indoor areas like rooms, buildings, cars, machines, etc., and deodorization of garbage collection and disposal systems in residential and commercial buildings.

The efficacy of ozone's application lies in the fact that it can reach hard-to-reach surfaces and spaces, while being easy to deploy, and suitable for use in virtually any surface and in the air inside a confined space. For these reasons, ozone can be used where other methods (e.g. Ultra-Violet (UV) light, alcohol, disinfecting chemicals, etc.) are not applicable.

Recently, the Covid-19 (SARS-CoV-2) pandemic that emerged in late 2019 requires effective solutions to stop its spread worldwide. The safe use of ozone indoors is the most effective way to combat the spread of the pandemic. Ozone is a powerful oxidizing agent and there are no viruses and bacteria resistant to it. Ozone instantly destroys the virus envelope without leaving the possibility for its restoration.

The SARS-CoV-2 pandemic has impacted public health and resulted in the loss of trillions of dollars from the global economy. Therefore, safe antimicrobial agents that suppress SARS-CoV-2 transmission are needed; low-dose ozone gas is one such agent, together with alcohol, ultraviolet (UV) light, detergents, and sodium hypochlorite. However, liquid agents (alcohol, detergents, and hypochlorite) are effective against SARS-CoV-2, but leave water and cannot be used against airborne virus. UV can kill viruses in the air and on surfaces. However, UV light cannot penetrate complex 3D objects so is ineffective for sterilizing them. High-dose ozone gas kills airborne SARS-CoV-2 and can be used to disinfect complex objects but is also toxic. Low-dose ozone gas requires high humidity for maximum effectiveness, but is nondestructive, nontoxic, and effective against airborne and fomite-borne SARS-CoV-2. Therefore, the SARS-CoV-2 disinfection method should be selected according to the specific situation.

Low-dose ozone gas could prove very useful in hospitals and hotels, where large numbers of people congregate continuously. The structure of hospital and hotel rooms is complex, and they contain desks, sofas, personal computers, air conditioners, medical devices, testing equipment, and bedding. In addition, the presence of SARS-CoV-2, in the air or on objects, is more likely in hospitals than in other settings. In combination with other precautions, such as frequent hand washing with detergent, wiping with alcohol, and UV irradiation, low-dose ozone gas and a humidifier will decrease the likelihood of infection. Also, low-dose ozone gas will likely be effective in other settings, including offices, schools, cars, ambulances, restaurants, bars, and museums.

However, besides its advantages and efficacy as a disinfecting and deodorization agents, high concentrations of ozone pose serious health risks to human. This is a very serious risk and at the same time a very important demotivation for the use of ozone in indoor areas. Currently, there are no known examples of ozone systems for indoor use, which can prove that they alleviate risk to humans frequenting the ozone rich indoor areas. For this reason, the widespread use of ozone in indoor areas is practically non-existent. An even more cumbersome situation exists in public indoor areas (e.g. offices, public transport, etc.) and especially hospitals. As these public areas are frequented by large numbers of people, the need for efficient disinfection of surfaces and air is imperative but is not adequately addressed for the reasons previously explained. Hospitals are the most critical of all indoor spaces, as they house very large concentrations of human carriers of the SARS-CoV-2 and many patients who are extremely vulnerable to infection. It is in hospitals that the need for a very efficient, easy-to-use, cheap and fast disinfection solution is needed, which will not introduce new health risks.

Although people have experimented with ozone, alone or in combination with other disinfection and odorization agents, there is currently no solution on how to regulate the use of ozone so that it is both efficient for the oxidation of bacteria and viruses, and not harmful to humans.

There is, therefore, a need for a solution to the problem of efficiently disinfecting and deodorizing indoor areas with ozone, without introducing health risks to the people inside these areas.

### SUMMARY

The present invention solves the problem of efficiently disinfecting and deodorizing indoor areas with ozone, without introducing health risks to the people inside these areas. The invention solves the problem using a multi-tiered control mechanism which ensures uninterrupted safety monitoring and operation of the proposed system by exploiting sensor readings to determine malfunctions and dangerous events, and time schedules, for enforcing automatic and immediate inhibition of the system operation.

In a first exemplary embodiment, a cold plasma ozone generator is used to create ozone from oxygen in atmospheric air provided under pressure by a compressor. The ozone is then distributed through a series of ozone tubes to one or more rooms where disinfection and/or deodorization is needed. The flow of ozone in the ozone tubes is regulated by valves, each valve being connected to one tube, and which valves are controlled by control signals generated from a main control module and relayed to the valves by the ozone generator unit. The main control module collects data from ozone concentration and IR motion detectors installed at the rooms where the ozone is distributed, temperature and high-voltage sensors in the ozone generator, and pressure sensors in the ozone distribution system (i.e. the ozone tubes and connected components), and uses a schedule associating information derived from the sensory data, with dates, time slots, and desired ozone concentration, to create control signals for controlling the generation and distribution of ozone for meeting safety and environmental standards. If ozone concentrations in excess of predefined levels are detected, ozone generation and/or distribution is interrupted, while when hardware or software malfunction of the system components are detected the main control module may command either the system to enter in emergency shutdown or lockdown, or the local control module or the ozone generator takes control of the generation and distribution of ozone. A combination of GPRS, ZigBee, and WiFi networks are used to connect the system components to each other. The system can supply ozone to 4 rooms, one at a time.

In a second exemplary embodiment, alternative network types are used for the network connections between the system components.

In a third exemplary embodiment, the system can supply ozone to less or more than 4 rooms.

In a fourth exemplary implementation, the system can supply ozone to more than one room at the same time.

Innovation lies in the multi-tiered control of a known type of ozone generator in combination with distribution and control equipment exploiting multi-sensory data and schedules for meeting safety and environmental standards.

The present innovative solution does not fall in the category of abstract idea as it discloses and claims a novel hardware and software system and a method for operating it.

### BRIEF DESCRIPTION OF THE DRAWINGS

FIG. 1 shows an exemplary high-level hardware architecture of the present innovative system.

FIG. 2 shows an exemplary high-level software control architecture of the present innovative system.

FIG. 3 shows an exemplary flowchart of the normal operation mode of the present innovative system.

FIG. 4 shows an exemplary flowchart of normal operation using a schedule.

FIG. 5 shows an exemplary flowchart of setting a schedule and staring the normal operation mode.

FIG. 6 shows a flowchart of emergency shutdown mode.

FIG. 7 shows a detailed flowchart of the system's operation.

### DETAILED DESCRIPTION

Any embodiment described herein as “exemplary” is not necessarily to be construed as preferred or advantageous over other embodiments.

The term “exemplary” is used herein to mean “serving as an example, instance, or illustration”.

The acronym “ASIC” is intended to mean “Application-Specific Integrated Circuit”.

The acronym “CD” is intended to mean “Compact Disc”.

The acronym “DSL” is intended to mean “Digital Subscriber Line”.

The acronym “DVD” is intended to mean “Digital Versatile Disc”.

The acronym “GPRS” is intended to mean “General Packet Radio Service”.

The acronym “GUI” is intended to mean “Graphical User Interface”.

The acronym “IP” is intended to mean “Internet Protocol”.

The acronym “IR” is intended to mean “Infra-Red”.

The acronym “ppm” is intended to mean “parts per million”.

The acronym “QR” is intended to mean “Quick Response”.

The acronym “S/W” is intended to mean “SoftWare”.

The acronym “THRES” is intended to mean “THREShold”.

The acronym “UV” is intended to mean “Ultra Violet”.

The acronym “WiFi” is intended to mean “Wireless Fidelity”.

The acronym “XML” is intended to mean “eXtensible Markup Language”.

The term “mobile device” may be used interchangeably with “client device” and “portable device with wireless capabilities”.

The term “user” may be used interchangeably with “regular user”, “ordinary user”, and “client”. It may also be used to mean “user of an application” or “user of a service”. It may also be used to refer to a “patient”, or to a “doctor”, unless otherwise explicitly stated or implicitly hinted at in the description, or obvious to a reader of ordinary skill in related art that these terms refer to different things, as this is apparent by the context of the discussion in which they appear.

The term “physician” may be used interchangeably with “doctor”.

The term “system” may be used interchangeably with “device”, “computing device”, “apparatus”, “computing apparatus”, and “service”, except where it is obvious to a reader of ordinary skill in related art that these terms refer to different things, as this is apparent by the context of the discussion in which they appear. Under any circumstance, and unless otherwise explicitly stated or implicitly hinted at in the description, these six terms should be considered to have the broadest meaning i.e. that of encompassing all six.

The term “module” may be used interchangeably with “unit” or “subunit”, except where it is obvious to a reader of ordinary skill in related art that these terms refer to different things, as this is apparent by the context of the discussion in which they appear.

The term “sensor” may be used interchangeably with “detector”.

The term “indoor” may be used interchangeably with “room”.

The terms “antenna”, “antennae”, “transducer”, “communications transducer”, and “data network” may be individually or collectively referred to as “means for communication”.

The term “module” may be used interchangeably with “unit” or “subunit”, “submodule”, and “component” except where it is obvious to a reader of ordinary skill in related art that these terms refer to different things, as this is apparent by the context of the discussion in which they appear.

System Hardware Architecture

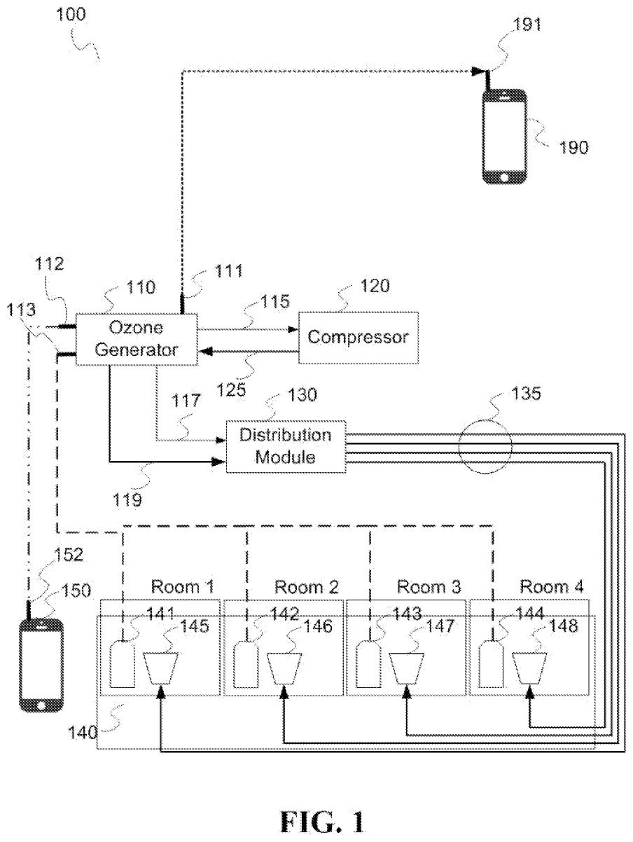

FIG. 1 shows an exemplary high-level hardware architecture of the present innovative system. System 100 is made up of four main entities. A room sub-system 140, which is made up of modules that are installed at the indoor areas where disinfection and odorization is needed, an ozone generation and distribution sub-system 105, which is connected to sub-system 140 and may be positioned at an area near the room where room sub-system 140 is installed (e.g. a secure, restricted-access room), a local control module 150 physically located to the same premises (e.g. the same building or campus) as ozone generations and distribution sub-system 105, and a main control module 190 which is typically located away from sub-systems 105, 140, 150 (e.g. at a service provision company offering ozone disinfection and deodorization services, or at a main control center for several ozone system installations at various sites, anywhere in the world).

Ozone generation and distribution sub-system 105 has an ozone generator 110, connected to a compressor 120 via a power control wire 115 and an air tube 125, and to a distribution module 130 via distribution control wire 117 and an ozone tube 119.

In one exemplary implementation, ozone generator 110 has a capacity of 10 g of ozone per hour with ozone generation on the principle of cold plasma. In alternative exemplary implementations, ozone generator 110 may have a higher or lower capacity for ozone generation, based on the intended use (i.e. the number and size of the indoor areas that need to be disinfected and deodorized).

Ozone is produced from atmospheric air using a pulsed streamer discharge with the parameters of a low-temperature plasma. Such a discharge produces an amount of energy sufficient for the dissociation of oxygen molecules, but insufficient for the destruction of nitrogen molecules. Thus, the very possibility of the formation of nitrogen oxides and, therefore, acids and an acidic film both on the inner surface of the chamber of ozone generator 110, distribution module 130, ozone tube 119, and output ozone tubes 135, and in emissions into the atmosphere is eliminated, which excludes adverse effects on the environment and system 100.

Air compressor 120 supplies fresh air to ozone generator 110 and is controlled by a control signal received from ozone generator 110 via power control wire 115. After receiving the control signal, from ozone generator 110, compressor 120 generates compressed air and feeds it to ozone generator 110 via air tube 125.

Ozone generator 110 receives the compressed air from compressor 120 and feeds it to its interior at a generation chamber where ozone is produced using the principle of cold plasma, which in well-known in the prior art.

Distribution module 130 receives a distribution control signal from ozone generator 110, via distribution control wire 117, and based on this signal it selectively opens and closes a set of valves (e.g. solenoid-magnetic, electrical, electropneumatic, etc. valves) thereby controlling which of the one or more output ozone tubes 135 will be shut-off and which will be opened for supplying an air-ozone mixture, received from ozone generator 110 via ozone tube 119, to the rooms where output ozone tubes 135 are installed and connected to respective, optional, ozone diffusers for distributing the same ozone concentration over the entire area of each room selected for disinfection and deodorization.

Tubes 119, 135 are resistant to ozone and may be selected from any such specimen known in prior art.

Inside the rooms, ozone concentration sensors 141, 142, 143, 144 are installed, together with Infra-Red (IR) sensors 145, 146, 147, 148 which are used to detect the presence of humans (by detecting motion). In one aspect, each ozone concentration sensor 141, 142, 143, 144 and Infra-Red (IR) sensor 145, 146, 147, 148 are integrated in a single device, while in another aspect they are separate devices.

Inside each room connected to tubes 135 is located a communications antenna, connected to an ozone concentration sensor 141, 142, 143, 144, and to an IR sensor 145, 146, 147, 148, respectively, for transmitting ozone concentration and motion detection signals containing readings sensed by ozone concentration sensors 141, 142, 143, 144 and IR sensors 145, 146, 147, 148. In one aspect, ozone concentration sensors 141, 142, 143, 144 and IR sensors 145, 146, 147, 148, are smart sensors with integrated electronics to pre-conditions and/or digitize the respective sensor readings, drive an integrated wireless (or in another aspect wired) transducer to feed a modulated signal to respective antennae connected to the sensor electronics. In another aspect, ozone concentration sensors 141, 142, 143, 144 and IR motion sensors 145, 146, 147, 148 are basic sensors, which are connected to a respective external processing module (not shown) and which is designed to perform the necessary pre-conditioning and/or digitization of the sensed signals and their transmission via the respective antennae.

In the present exemplary implementation, ozone concentration sensor 141, 142, 143, 144 and IR motion sensor 145, 146, 147, 148 signals are wirelessly transmitted via the ZigBee standard to a matching antenna 113 at ozone generator 110. The sensors are powered either from ordinary mains sockets (in the case of smart sensors), or are supplied with power by a special power socket in the electronics module where they are connected, or from an integrated battery.

Once connected to power, sensors 141-148 automatically connect to ozone generator 110 via a ZigBee wireless link (or in alternative implementations via any wireless or wired link) and are immediately ready to send signals (i.e. sensed data) to ozone generator 110. In the present exemplary implementation, sensors 141-148 send data every 5 seconds, while in alternative exemplary implementations they may send data at any chosen interval. The choice of the ZigBee connection is made so that the link between ozone generator 110 and sensors 141-148 is autonomous and does not depend on external and internal Internet networks, and thus can be guaranteed to feed sensed data to ozone generator 110 even if other available data networks are down (this is important as the overall system 100 control is done by the remotely located main control module 190; if the communication between main control module 190 and ozone generator 110 is interrupted, subsystems 105, 140, 150 will continue to operate with either local control module 150 or ozone generator 110 in control.

In alternative exemplary implementations other known wireless standards could be used, or even a wired communication standard could be used if ozone concentration sensor 141, 142, 143, 144 and IR motion sensor 145, 146, 147, 148 are wired to ozone generator 110.

Ozone generator 110 is also connected to main control module 190. In the present exemplary implementation, the connection is wireless, via the General Packet Radio Service (GPRS) protocol (including a built-in SIM modem and a SIM card that is not shown, and an GPRS antenna 111), while in alternative exemplary implementations any other type of wireless or wired network may be used). Main control module 190 is the highest-level controller of system 100. It receives sensor data from ozone generator 110 and processes them according to a software it runs for creating control signals that are then sent to ozone generator 110 for controlling the generation and distribution of ozone to the rooms for disinfection and deodorization.

Ozone generator 110 is also connected to local control module 150. In the present exemplary implementation, the connection is wireless, via a Wireless Fidelity (WiFi) protocol and a WiFi antenna 112), while in alternative exemplary implementations any other type of wireless or wired network may be used). Local control module 150 is the second highest-level controller of system 100. It receives sensor data from ozone generator 110 and processes them according to a software it runs for creating control signals that are then sent to ozone generator 110 for controlling the generation and distribution of ozone to the rooms for disinfection and deodorization.

In an alternative exemplary implementation, a single antenna may be used at ozone generator 110 for replacing two or three of the GPRS 111, WiFi 112, and ZigBee 113 antennae.

In accordance with current legislation in several countries, an ozone concentration of up to 0.1 ppm is considered safe for humans. System 100 according to the present embodiment is designed to meet this requirement and at the same time introduces an innovative multi-tiered (e.g., 3-tier) control mechanism (ozone generator 110, local control module 150, main control module 190) that exploits data from four types of sensors (ozone concentration sensors 141-144, IR motion detector sensors 145-148, pressure sensor(s), and high-voltage sensor). The control mechanism uses a combination of software and hardware to regulate ozone concentration to within the legal limit when humans are present in the indoor area, and provide higher concentrations when human are not present. Human presence is detected with IR sensors 145-148. System 100 is also designed to set and use a schedule, where the ozone provision and the regulation of the ozone concentration can be done individually per indoor area (i.e. per room) and per time and day, so as to anticipate human presence or absence, which is then verified by IR sensors 145-148.

As a result, system 100 ensures that its operation will never result in higher ozone concentrations when humans are present in indoor areas. Ozone concentration can be maintained within other higher limits in the absence of humans using software executed by the 3-tier control mechanism. For this reason, the software also provides two or more additional modes for regulating the ozone concentration.

At the same time, the 3-tier control mechanism uses HV sensor readings to ensure the correct ozone generation without nitrogen oxide byproducts, and pressure readings at one or more points in the ozone distribution tubes so as to ensure that the correct supply of ozone-air mixture is maintained at all times and only where intended, according to the schedule. The main control is done by main control module 190, which (using antenna 191) is in constant communication with ozone generator 110. When this communication is lost, local control module 150 and/or ozone generator 110 takes over control. Local users or administrators may also monitor and control the operation of ozone generator 110 via local control module 150, while manual override of automatic operation is also possible by optional buttons (or a Graphical User Interface (GUI)) installed on the outside of ozone generator 110 when the connection between main control module 190 and ozone generator 110 is lost.

The present system 100 consisting of ozone generator 110, operating on the principle of cold plasma, and combined with infrared motion detectors located in each room, and an ozone delivery system to the treatment site can maintain a predetermined concentration of up to 0.1 ppm, which the safety level when humans are present in the room.

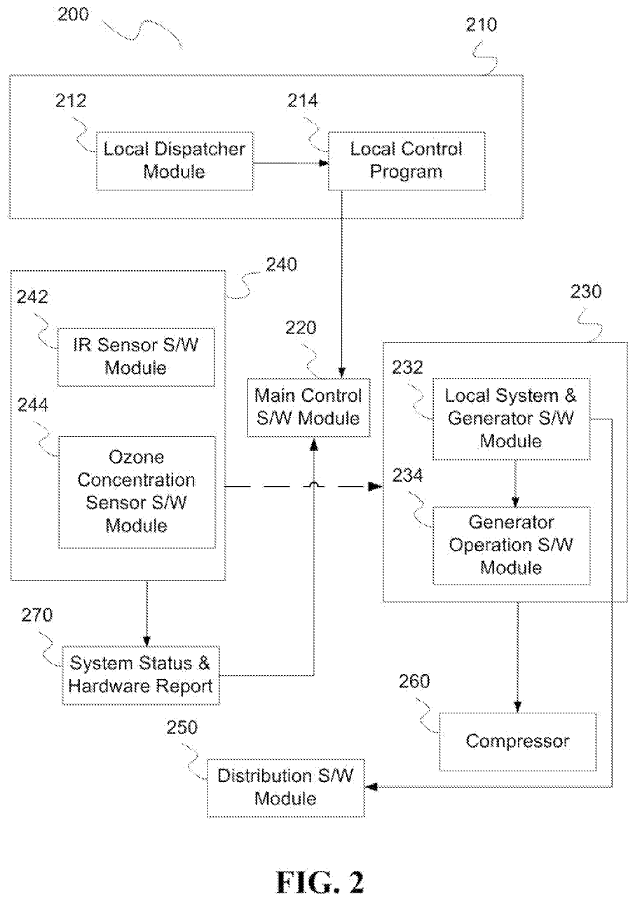

FIG. 2 shows an exemplary high-level software control architecture of the present innovative system. SoftWare (S/W) architecture 200 has a local control S/W module 210, a main control S/W module 220, an ozone generator S/W module 230, and a monitoring system S/W module 240. Modules 210-240 are executed on hardware modules 150, 190, 110, and 140, respectively and may be implemented in any programming language (including descriptive languages like eXtensible Markup Language (XML)) as a software, package, function, routine, firmware and the like.

Local control S/W module 210 has, in one aspect, a local dispatcher module 212 which is responsible for installing and setting a local control program 214. After a specialist of the provider has installed and set local control program 214 at the customer's site (i.e. at local control module 150), following installation of system 100, the provider of disinfection and deodorization services (who may for example lease system 100 and its software 200 to the customer, e.g. a hospital, hotel, etc.) the customer gains access to the control of ozone generation and distribution sub-system 105 and may proceed to set a new schedule or select (and optionally adapt) an existing schedule preloaded with local control program 214. The schedule among other data, contains ozone concentrations per working time/date and per room. The schedule (or its modifications) is then sent to main control S/W module 220 via a data network. In the present exemplary implementation, local control module 150, which executes S/W 210, transmits via a WiFi connection the schedule (or its modifications) to ozone generator S/W module 230, which is executed at ozone generator 110, and ozone generator S/W module 230 simply relays using a GPRS link the schedule to main control S/W module 220, which is executed on main control module 190.

Main control S/W module 220 receives the schedule and processes it to create commands for transmission, via the GPRS network, to ozone generator S/W module 230. Upon receipt of command(s) from main control S/W module 220, and in particular by its local system and generator S/W module 232, ozone generator S/W module 230 is awaken and enters normal operating mode, triggering with a set of control signals its generator operation S/W module 234, to take control of the operation of compressor 120, which executes compressor S/W module 260.

Local system and generator S/W module 232 also controls distribution module 130. In one implementation, distribution module 130 is a smart distribution module and executes distribution S/W module 250, which accepts control signals from local system and generator S/W module 232, actuates the valves connected to ozone distribution tubes 135, and relays pressure sensor readings to local system and generator S/W module 232.

In an alternative exemplary implementation, distribution module 130 is a basic electromechanics module (not a smart one, therefore, it does not execute distribution S/W module 250) which receives signals (e.g. voltage signals) which actuate its valves (e.g. magnetic valves, electromechanical valves, etc.)

In alternative exemplary implementations different network types may be used. In other alternative exemplary implementations, local control program 214 sends the schedule directly to main control S/W module 220 and to ozone generator S/W module 230.

Local system and generator S/W module 232 also receives HV sensor ***** signals, and signals from room sub-system 140, which executes monitoring system S/W module 240, which handles the communication and manipulation of sensor data. Monitoring system S/W module 240 has an IR motion sensor S/W module 242, and an ozone concentration sensor S/W module 244, which perform pre-processing, calibration, etc. operations on the data sensed by IR 145-148 and ozone concentration 141-144 sensors, respectively.

Monitoring system S/W module 240 also creates periodic system status and hardware reports 270, which it transmits (via ZigBee in the present exemplary implementation or via other network types in alternative exemplary implementations) to main control S/W module 220. Main control S/W module 220 processes system status and hardware reports 270, and if anomalies in the status and operation of any component of system 100 is detected, then main control S/W module 220 commands ozone generator S/W module 230 to enter either in emergency shutdown mode or in emergency lockdown mode.

Under normal operating mode, each customer may use local control module 150 (e.g. a local computing apparatus) to access at main control module 190, an instance of main control S/W module 220, which is associated only with the ozone generation and distribution sub-system 105, room sub-system 140, and local control module 150 installed at the customer's premises. This instance is configured according to the schedule sent to it by local control program 214. Customers cannot access other instances unless they have additional installations of system 100 and they are authorized to access them; in such a case, the different instances of main control S/W module 220 and the corresponding schedules are siloed by main control module 190 and their data and control functionality and commands are always separate and dispatch only to the associated hardware and software modules of the corresponding installed system 100.

In the above-described software architecture 200, the state of the hardware modules of system 100 is controlled by local control program 214 and recorded by main control S/W module 220 at main control module 190. These recorded data are kept in digital storage, so that if liabilities and legal or insurance disputes arise they may be used as proof of the correct operation and control of system 100. These records may be supplemented with historical data on human interventions on system 100, times schedules, etc. In the present exemplary implementation, sensor data is collected logged every 5 sec but other intervals may be used in alternative exemplary implementations.

Normal Operation Mode

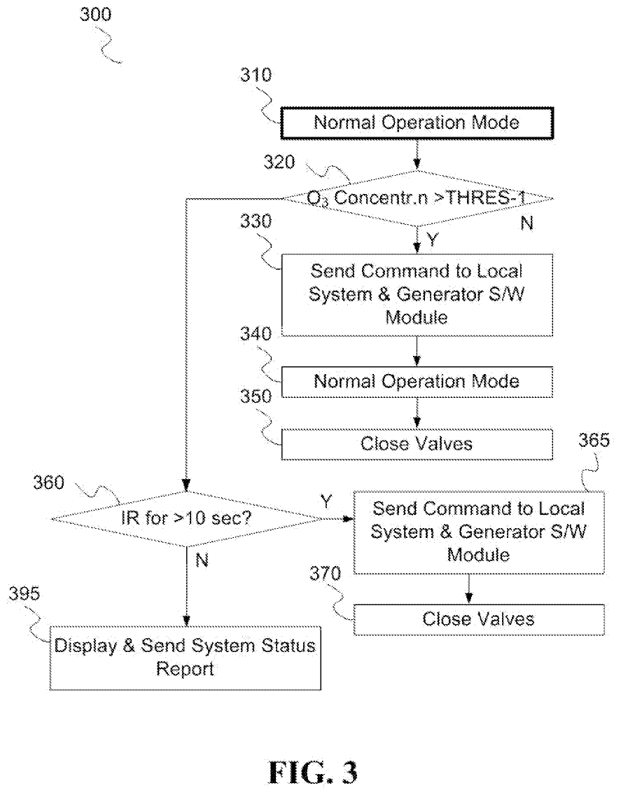

FIG. 3 shows an exemplary flowchart of the normal operation mode of the present innovative system. Flowchart 300 is implemented by main control S/W module 220, which initially commands local system and generator S/W module 232 to enter normal operation mode 310; it is assumed that all hardware modules of system 100 are powered on. If the last reading 320 from any of ozone concentration sensors 141-144, that is received at main control S/W module 220, is above a threshold 320, THRES-1, main control S/W module 220 sends a command 330 (or control code or signal) to local system and generator S/W module 232, which then sends a command (or control code or signal according to the type of distribution module 130) to distribution S/W module 250 for closing the valve(s) 340 connected to output ozone tubes 135 and fitted to the same room with the ozone concentration sensor(s) that sensed the corresponding readings that exceeded THRES-1. This way, ozone supply to any room where the ozone concentration is above the threshold value THRES-1 is immediately and automatically interrupted. THRES-1 takes its value from the corresponding entries in the schedule held by main control S/W module 220, local control program 214, and ozone generator 110. So, THRES-1 may be assigned different values for the same room and for different days, times, and instances where a human is present or absent from the room where the ozone is supplied.

In the present exemplary implementation, after the valve connected to a tube 135 supplying ozone to a room has been closed, another valve connected to a tube 135 supplying ozone to another room is opened. As a result, ozone supply is provided at one room at a time 350, sequentially until all rooms have been supplied with ozone; the transition from one room to the next is done either at the expiry of the time duration of ozone supply to one room, or after the ozone supply in the same room was interrupted as a result of sensed high ozone concentration above THRES-1, or after a user interrupt (i.e. manual override).

In alternative exemplary implementations, it is possible to supply ozone to more than one rooms at any time.

If the last reading 320 from any of ozone concentration sensors 141-144, that is received at main control S/W module 220, is not above threshold 320, THRES-1, main control S/W module 220 checks if a reading from any IR sensor 145-148 indicates motion for 10 sec or more 360, which it then interprets as an indication of human presence in the room where the corresponding IR sensor is installed.

If human presence is detected in a room, main control S/W module 220 sends a command 365 (or control code or signal) to local system and generator S/W module 232, which then sends a command (or control code or signal according to the type of distribution module 130) to distribution S/W module 250 for closing the valve(s) 370 connected to output ozone tubes 135 and fitted to the same room with the IR sensor(s) that sensed human presence (i.e. motion for 10 sec or more). This way, ozone supply to any room where a human is present, is regulated to the allowable maximum of 0.1 ppm by immediately and automatically interrupting ozone supply. Ozone concentration in the room then drops and ozone supply may then, optionally, be intermittently restored to keep the ozone concentration below THRES-1 but at an optional tolerance predefined in the schedule.

If a reading from any IR sensor 145-148 does not indicate motion for 10 sec or more 360, which main control S/W module 220 then interprets as an indication of no human presence in the room where the corresponding IR sensor is installed, main control S/W module 220 displays system status and also sends 395 a system status report to local control program 214, and allows normal operation mode 310 to continue.

Example Normal Operation Mode using a Schedule

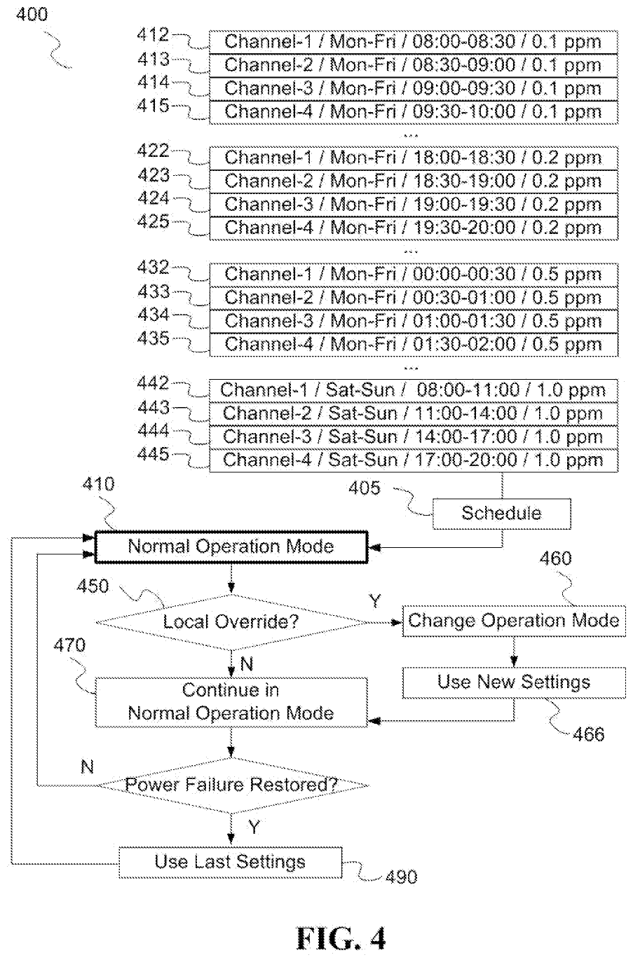

FIG. 4 shows an exemplary flowchart of normal operation using a schedule. Flowchart 400 is implemented by main control S/W module 220, which initially commands local system and generator S/W module 232 to enter normal operation mode 410 based on a selected or edited schedule 402; it is assumed that all hardware modules of system 100 are powered on. Schedule 402 contains an association of channels (i.e. corresponding to output ozone tubes 135) with days, time slots (or zones), ozone concentrations, and IR sensors 145-148 and ozone concentration sensors 141-144 (which are located in the rooms where ozone is fed by the respective channels). Optionally, schedule 402, may contain Internet Protocol (IP) addresses of the hardware elements of system 100, credentials of the customer or operator who edited the schedule, time stamped schedule version, etc.

In the present exemplary schedule 402, 4 channels are used and associated with a first set of time slots:
channel_1 412 is associated with weekdays (e.g. Monday-Friday), time slot (08:00-08:30), ozone concentration of 0.1 ppm, IR sensor 145, and ozone concentration sensor 141 (i.e. with a first room)
channel_2 413 is associated with weekdays (e.g. Monday-Friday), time slot (08:30-09:00), ozone concentration of 0.1 ppm, IR sensor 146, and ozone concentration sensor 142 (i.e. with a second room)
channel_3 414 is associated with weekdays (e.g. Monday-Friday), time slot (09:00-09:30), ozone concentration of 0.1 ppm, IR sensor 147, and ozone concentration sensor 143 (i.e. with a third room)
channel_4 415 is associated with weekdays (e.g. Monday-Friday), time slot (09:30-10:00), ozone concentration of 0.1 ppm, IR sensor 147, and ozone concentration sensor 143 (i.e. with a fourth room)

During the above time slots, human presence is expected in the four rooms and, therefore, the maximum ozone concentration is set to the 0.1 ppm safe level.

The same four channels are also assigned a second set of time slots, and ozone concentrations. For example:
channel_1 422 is associated with weekdays (e.g. Monday-Friday), time slot (18:00-18:30), ozone concentration of 0.2 ppm, IR sensor 145, and ozone concentration sensor 141 (i.e. with a first room)
channel_2 423 is associated with weekdays (e.g. Monday-Friday), time slot (18:30-19:00), ozone concentration of 0.2 ppm, IR sensor 146, and ozone concentration sensor 142 (i.e. with a second room)
channel_3 424 is associated with weekdays (e.g. Monday-Friday), time slot (19:00-19:30), ozone concentration of 0.2 ppm, IR sensor 147, and ozone concentration sensor 143 (i.e. with a third room)
channel_4 425 is associated with weekdays (e.g. Monday-Friday), time slot (19:30-20:00), ozone concentration of 0.2 ppm, IR sensor 147, and ozone concentration sensor 143 (i.e. with a fourth room)

The times slots (18:00-20:00) are associated with no human presence, or with very brief occasional presence (e.g. a few seconds to pick a patient's file from an examination office at a hospital) and, therefore, the maximum ozone concentration is set to 0.2 ppm. This higher ozone concentration may be used to get more efficient disinfection and deodorization, and while being above the 0.1 ppm safety threshold, it is still low enough to allow fast drop below the safety threshold (e.g. within a few seconds after the emergency shutdown of the ozone provision to the room) if the occasional human presence in the room turns out to be longer than 10 sec.

In an alternative exemplary implementation, different ozone concentrations may be set for each channel. For instance, if room 4 is locked and marked not for access for the time slot (19:30-20:00), ozone concentration of 0.3 ppm can be set. This is higher above the safety threshold for more efficient disinfection because room 4 may have been seriously infected due to an accident, but still not very high in the unlikely event that a member of staff invades room 4 despite the prohibitive signage. Other possible scenarios may exist. If the staff does invade room 4, ozone supply to room 4 is interrupted to allow ozone concentration to (rather) quickly drop to the safe 0.1 ppm level.

The same four channels are also assigned a third set of time slots, and ozone concentrations. For example:
channel_1 432 is associated with weekdays (e.g. Monday-Friday), time slot (00:00-00:30), ozone concentration of 0.5 ppm, IR sensor 145, and ozone concentration sensor 141 (i.e. with a first room)
channel_2 433 is associated with weekdays (e.g. Monday-Friday), time slot (00:30-01:00), ozone concentration of 0.5 ppm, IR sensor 146, and ozone concentration sensor 142 (i.e. with a second room)
channel_3 434 is associated with weekdays (e.g. Monday-Friday), time slot (01:00-01:30), ozone concentration of 0.5 ppm, IR sensor 147, and ozone concentration sensor 143 (i.e. with a third room)
channel_4 435 is associated with weekdays (e.g. Monday-Friday), time slot (01:30-02:00), ozone concentration of 0.5 ppm, IR sensor 147, and ozone concentration sensor 143 (i.e. with a fourth room)

The times slots (00:00-02:00) are associated with night hours when no human presence is planned or expected as it is out of working hours and, therefore, the maximum ozone concentration is set to 0.5 ppm. This higher ozone concentration may be used to get the highest level of efficient disinfection and deodorization.

The same four channels are also assigned a fourth set of time slots, and ozone concentrations. For example:
channel_1 442 is associated with weekends (i.e. Saturday-Sunday), time slot (08:00-11:00), ozone concentration of 1.0 ppm, IR sensor 145, and ozone concentration sensor 141 (i.e. with a first room)
channel_2 443 is associated with weekends (i.e. Saturday-Sunday), time slot (11:00-14:00), ozone concentration of 1.0 ppm, IR sensor 146, and ozone concentration sensor 142 (i.e. with a second room)
channel_3 444 is associated with weekends (i.e. Saturday-Sunday), time slot (14:00-17:00), ozone concentration of 1.0 ppm, IR sensor 147, and ozone concentration sensor 143 (i.e. with a third room)
channel_4 445 is associated with weekends (i.e. Saturday-Sunday), time slot (17:00-20:00), ozone concentration of 1.0 ppm, IR sensor 147, and ozone concentration sensor 143 (i.e. with a fourth room)

The times slots (08:00-20:00) are associated with weekends when no human presence is planned or expected as it is out of working hours and working days (house-keeping and cleaning is only performed on weekdays), therefore, the maximum ozone concentration is set to 1.0 ppm. This is the highest ozone concentration used and is only reserved for weekends when human absence is guaranteed.

In alternative exemplary implementations, less or more channels may be used to supply ozone to less or more than 4 rooms, and different ozone levels may be set for each channel per timeslot set, while more or less sets of time slots may be allocated.

Main control S/W module 220, processes schedule 402 to produce and transmit a set of commands for controlling the operation of the other components of system 100, and also transmits the schedule (after verifying it) to local control program 214 and to local system and generator S/W module 232, or compile a software package (e.g. an executable program, or an XML description, or a script file, or the like) and transmit it alone or with the schedule so that local control program 214 and/or local system and generator S/W module 232 can use the received schedule and/or compiled software package to take control and operate components 105 without using main control S/W module 220. This scenario is useful if the network connection between ozone generator 110 and main control module 190 is interrupted.

During normal operation mode 410, main control S/W module 220 and local control program 214 may display ozone concentration levels at each room (associated with the set channels), system operational parameters and status, and the used schedule.

Local control program 214 checks for local overrides 450 entered by the customer either through a GUI provided by local control program 214, or directly at ozone generator 110 via button or an integrated GUI. If a local override 450 is detected, it is sent by local control program 214 to main control S/W module 220, which then commands local system and generator S/W module 232 to change operation mode 460 or to operate according to a new schedule or settings 466.

If a local override 450 is not detected, or if a change to a new operation mode 460, or with a new schedule or settings 466 is done, the new or adapted normal operation mode is executed 470.

Local control program 214 then checks for a power failure restoration event 480. If a power failure occurred and the operation is restored, the last used operating settings are used 490 and normal operation is executed 410. If no power failure occurred, the normal operating mode with the current settings continues 410.

Setting a Schedule and Staring the Normal Operation Mode

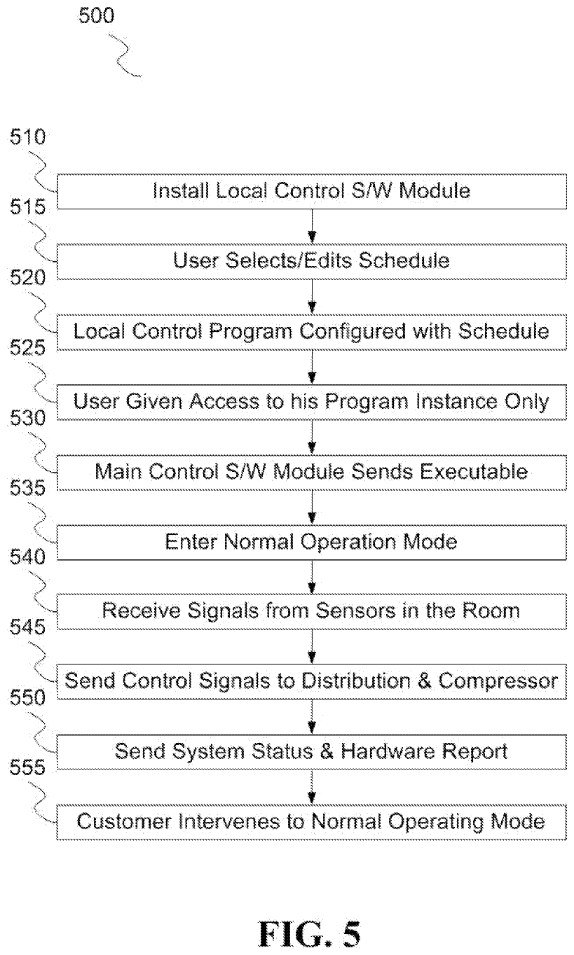

FIG. 5 shows an exemplary flowchart of setting a schedule and staring the normal operation mode. Flowchart 500 starts with a technical staff installing 510 local control S/W module 210 (i.e. local dispatcher module 212 and local control program 214). The customer selects or edits an available schedule 515 for the system installation at his site at local control S/W module 210. Local control S/W module 210 confirms 520 local control program 214 and/or schedule and sends it to at main control S/W module 220. The customer is given access 525 only to the instance of main control S/W module 220 that corresponds to the system installation at his site.

Main control S/W module 220 then sends 530 an executable program, or an XML description, or a script file, or the like) to local system and generator S/W module 232. After receiving the file, local system and generator S/W module 232 enters normal operation mode 535 and functions according to the software and schedule.

Local system and generator S/W module 232 receives 540 signals from ozone concentration 141-144 and IR sensors 145-148 and sends them to main control S/W module 220, which processes the sensor signals according to schedule 402, and replies with control signals (e.g. including time slots, ozone concentrations, etc.) to local system and generator S/W module 232, which in turns sends 545 the control signals to distribution S/W module 250 and compressor S/W module 260.

Local system and generator S/W module 232 then sends 550 a system status and hardware report 270 to main control S/W module 220.

The customer can control 555 and intervene in the normal operating mode of system 100 via main control S/W module 220, using local control program 214 to access main control S/W module 220.

Emergency Lockdown Mode

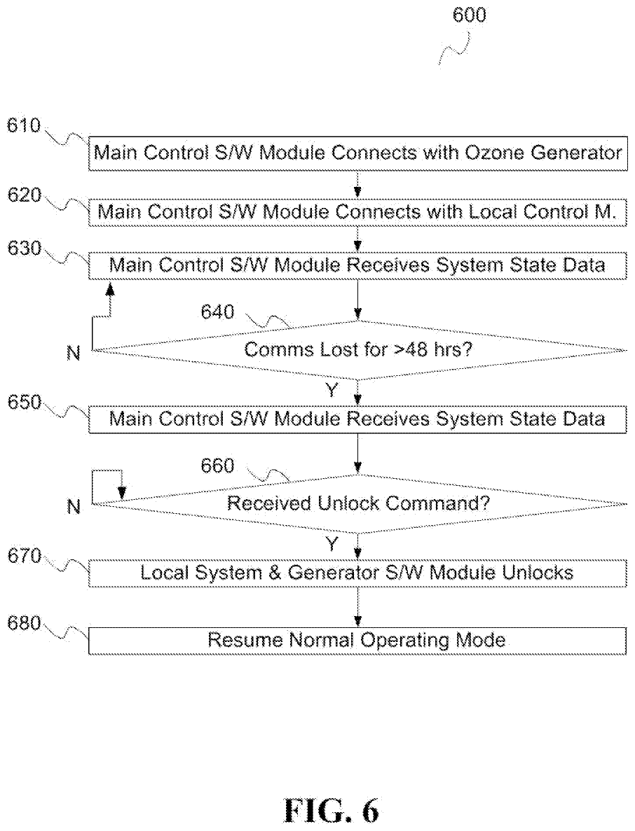

FIG. 6 shows a flowchart of emergency shutdown mode. Flowchart 600 starts with main control S/W module 220 and local system and generator S/W module 232 connecting 610 via a data connection (GPRS in the present exemplary implementation, or any type of network in alternative exemplary implementations). Main control S/W module 220 and local control program 214 connect 620 via local system and generator S/W module 232 (in the present exemplary implementation using the GPRS and a WiFi network connected back-to-back, or any other network combinations in alternative exemplary implementations).

Main control S/W module 220 receives 630 the communications state with all other system 100 components, data from local control program 214, data from local system and generator S/W module 232, and sensor data, and checks 640 if communication with any system 100 component is lost for more than 48 hours.

If communication is lost for less than 48 hours, local system and generator S/W module 232 takes control of the system 100 operation.

If communication is lost for more than 48 hours, generator has taken takes control of the system 100 operation and once communication resumes, main control S/W module 220 commands local system and generator S/W module 232 to enter emergency lockdown mode 650, which lock all system 100 components. Once system 100 has entered emergency lockdown mode, it can be unlocked only by the service provider with a command sent from his account at main control module 190 to local system and generator S/W module 232. Otherwise, methodology 600 loops step 640 until a positive result occurs.

If local system and generator S/W module 232 has received 660 an unlock command from the service provider's account at main control module 190, local system and generator S/W module 232 unlocks 670 and resumes normal operating mode 680, otherwise it keeps checking for the reception of an unlock command.

Detailed System Operation

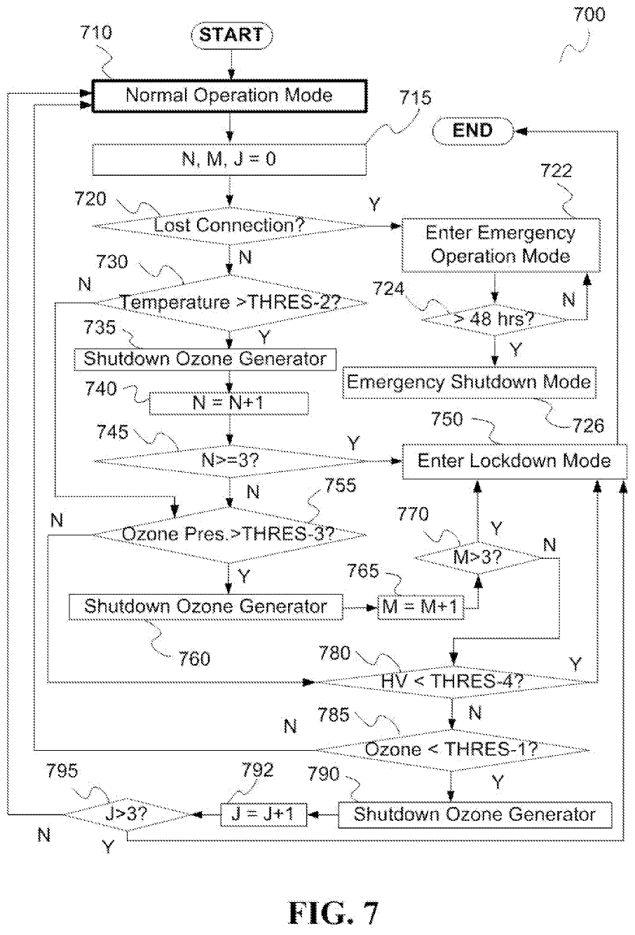

FIG. 7 shows a detailed flowchart of the system's operation. Flowchart 700 starts with system 100 entering normal operation mode 710. Counters N, M, J are each set to “0” 715. Main control S/W module 220 checks if network connection with ozone generator 110 and/or sensors 141-148 is lost 720, and if yes, commands ozone generator 110 to turn its local system and generator S/W module 232 and enter emergency operation mode 722. Main control S/W module 220 then checks if network connection is lost for more than 48 hours 724 and if yes it commands local system and generator S/W module 232 to enter emergency shutdown mode 726, or if no, local system and generator S/W module 232 remains in emergency operation mode 722.

If network connection is not lost 720, main control S/W module 220 checks if the temperature 730 of the generation chamber of ozone generator 110 has exceeded a second threshold THRES-2. If yes, it commands local system and generator S/W module 232 to switch-off 735 ozone generator 110, sets counter N=N+1 740, and checks 745 if N>=3 (i.e. the temperature of the generation chamber of ozone generator 110 has exceeded THRES-2 for a third time). It N>=3, then main control S/W module 220 commands local system and generator S/W module 232 to enter system lockdown mode 750. Unlocking can only be done by the only by the service provider with a command sent from his account at main control module 190 to local system and generator S/W module 232.

If temperature 730 of the generation chamber of ozone generator 110 has not exceeded THRES-2, flow jumps to step 755 (see below).

If N<3, then main control S/W module 220 checks if the pressure of the ozone-air mixture in the ozone distribution system has exceeded a third threshold THRES-3 755. If yes, main control S/W module 220 commands local system and generator S/W module 232 to command generator operation S/W module 234 to shutdown 760 ozone generator 110, sets counter M=M+1 765, and checks is M>=3 770, and if yes, main control S/W module 220 commands local system and generator S/W module 232 to enter system lockdown mode 750. If no, main control S/W module 220 checks if High Voltage (HV) at a HV transformer of ozone generator 110 has dropped below a fourth threshold THRES-4 780, and if yes main control S/W module 220 commands local system and generator S/W module 232 to enter system lockdown mode 750. If no, main control S/W module 220 checks if the ozone concentration at a room has exceeded the first threshold THRES-1 785. If the ozone concentration at a room has exceeded the first threshold THRES-1 785, main control S/W module 220 commands local system and generator S/W module 232 to command distribution S/W module 250 to close the respective valve, and, optionally, generator operation S/W module 234 to shutdown 790 ozone generator 110 by commanding it to enter emergency shutdown mode, and then sets counter J=J+1 792 and checks if J>3 795 (i.e. ozone concentration has exceeded three time the threshold THRES-1 for the same room). If J>3 795, main control S/W module 220 commands local system and generator S/W module 232 to enter system lockdown mode 750, while if not, normal operation mode 710 continues.

Similarly, if the ozone concentration at a room has not exceeded the first threshold THRES-1 785, normal operation mode 710 continues.

If the pressure of the ozone-air mixture in the ozone distribution system has not exceeded a third threshold THRES-3 755, the flow branches to step 780.

After either step 750 and 726, methodology 700 ends.

In an alternative exemplary implementation, modules 105 and 140 may be equipped with a position (e.g. Global Positioning System (GPS), Glonass, Galileo, indoor positioning system, etc.), movement sensor (e,g. inertia sensor, magnetic sensor, etc.) for detecting unauthorized movement of the hardware modules of system of system 100. If such a movement is detected, 110 ozone generator 110 (and in particular local system and generator S/W module 232) notifies main control S/W module 220 for security and safety reasons, and main control S/W module 220 also logs this event.

Depending on the particular implementation and operation mode, some or all of system's 100 modules may be shutdown.

System 100 ensures safe and uninterrupted operation by a 3-tier control mechanism using main control S/W module 220, local control program 214, and local system and generator S/W module 232. The control for obeying the safety limits during system operation is performed by software, firmware, ASICs, or their combination and is automatic, thereby preventing human error in the system control. To further minimize human error, manual control is disabled when main control module is connected with the other system modules.

Ozone generator 110 may, in one aspect, contain electronic logic to interface and/or filter, digitize, or process sensor readings from non-smart sensors.

In one aspect, if non-authorized interference with the system operation is detected, the 3-tier control mechanism restores operating parameters within a time interval (e.g. less than 10 mins in present exemplary implementation, or less in other exemplary implementations).

The computing apparatuses used in system 100 may be any type of purpose build electronic device implementing the present innovative solution, or a general-purpose computing apparatus (e.g. a server, desktop computer, laptop, smartphone, tablet, and the like) which runs special software that transforms it to an application-specific computing device adapted to implement the present innovative solution.

The steps of methodologies 300-700, are obviously exemplary and any person skinned in related art may add, remove, merge, or change the order of steps without departing from the scope of protection of the present innovative solution and without the need to have and apply inventive skill or undue experimentation.

The present innovative solution can be implemented by software written in any programming language, or in an abstract language (e.g. a metadata-based description which is then interpreted by a software or hardware component). The software running in the above-mentioned hardware, effectively transforms a general-purpose or a special-purpose hardware or computing device, apparatus or system into one that specifically implements the present innovative solution. In another aspect an embedded system is used for the wearable device.

Alternatively, the present innovative solution can be implemented in Application Specific Integrated Circuits (ASIC) or other hardware technology.

The above exemplary embodiment descriptions are simplified and do not include hardware and software elements that are used in the embodiments but are not part of the current invention, are not needed for the understanding of the embodiments, and are obvious to any user of ordinary skill in related art. Furthermore, variations of the described system architecture are possible, where, for instance, some servers may be omitted or others added.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of a preferred embodiment and best mode of the invention known to the applicant at this time of filing the application has been presented and is intended for the purposes of illustration and description. It is not intended to be exhaustive or limit the invention to the precise form disclosed and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated. Therefore, it is intended that the invention not be limited to the particular embodiments disclosed for carrying out this invention, but that the invention will include all embodiments falling within the scope of the appended claims.

In one or more exemplary embodiments, the functions described may be implemented in hardware, software, firmware, or any combination thereof. If implemented in software, the functions may be stored on or transmitted over as one or more instructions or code on a computer program product including a computer readable medium. Computer-readable media includes both computer storage media and communication media including any medium that facilitates transfer of a computer program from one place to another. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and blu-ray disc where disks usually reproduce data magnetically, while discs reproduce data optically with lasers. Combinations of the above should also be included within the scope of computer-readable media.

The previous description of the disclosed exemplary embodiments is provided to enable any person skilled in the art to make or use the present invention. Various modifications to these exemplary embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

1-20. (canceled)

21. A system for controlling a provision of an ozone for disinfecting an indoor area, the system comprising:
an ozone generator for generating an ozone-air mixture, wherein the ozone generator having an ozone generation chamber, a temperature sensor in the ozone generation chamber, a communication unit, and a high-voltage sensor;
a compressor connected to the ozone generator and configured to provide a compressed air to the ozone generator upon receipt of a power signal from the ozone generator;
a motion sensor connected to the ozone generator and configured for sensing a presence of a human in the indoor area;
an ozone concentration sensor connected to the ozone generator and configured for sensing an ozone concentration in the indoor area;
a distribution module having an input tube configured for receiving the ozone-air mixture from the ozone generator, an output tube connected to the indoor area, and an electrically controlled valve connected at the output tube, wherein the distribution module is configured for selectively distributing the ozone-air mixture to the indoor area according to a distribution control signal received from the ozone generator;
a pressure sensor connected to the ozone generator and configured for sensing a pressure of the ozone-air mixture;
a local control module comprising a first communication device configured to communicate with the communication unit of the ozone generator; and
a main control module comprising a second communication device connected with the ozone generator;
wherein the motion sensor and the ozone concentration sensor are connected to the ozone generator via a third communication device, and the local control module is configured to enter one of a normal operation mode, an emergency shutdown mode, and an emergency lockdown mode.

22. The system of claim 21, wherein the normal operating mode comprises executing a schedule configured for:
commanding the ozone generator to receive, process according to the schedule, and transmit to the local control module and to the main control module, signals from the temperature sensor, the high-voltage sensor, the motion sensor, and the ozone concentration sensor;
commanding the compressor to provide air to the ozone generator; and
commanding the distribution module to selectively distribute the ozone-air mixture to the indoor area according to the schedule and the signals from the temperature sensor, the high-voltage sensor, the motion sensor, and the ozone concentration sensor after receiving a signal from the main control module.

23. The system of claim 21, wherein the emergency shutdown mode is entered upon the ozone generator detecting one of a loss of connection between the main control module and the ozone generator, and a signal sourced from one of the ozone concentration sensor exceeding a first threshold, the temperature sensor exceeding a second threshold, the pressure sensor substantially differing from a third threshold, or the main control module detecting a problem with a predetermined component.

24. The system of claim 21, wherein the emergency lockdown mode is entered upon the ozone generator detecting one of a loss of connection between the main control module and the ozone generator, and a signal sourced from one of the ozone concentration sensor exceeding a first threshold for a first time period, the temperature sensor exceeding a second threshold for a second time period, the pressure sensor substantially differing from a third threshold for a third time period, the main control module detecting a problem with a predetermined component for a fourth time period, or the high-voltage sensor signaling a droppage below a fourth threshold.

25. The system of claim 21, wherein the ozone-air mixture is configured for deodorizing the indoor area.

26. The system of claim 21, wherein the indoor area comprises a plurality of indoor areas; and wherein the ozone-air mixture is configured for deodorizing the plurality of indoor areas.

27. The system of claim 21, wherein the motion sensor is an infra-red (IR) motion sensor.

28. The system of claim 21, wherein the first communication device, the second communication device, and third communication device are selected to respectively include from at least one of a first, a second, and a third antenna, a first, a second, and a third communications transducer, and a first, a second, and a third data network.

29. The system of claim 28, wherein the first antenna is a Wireless Fidelity (WiFi) antenna, the second antenna is a General Packet Radio Service (GPRS) antenna, and the third antenna is a ZigBee antenna.

30. The system of claim 21, wherein the ozone generator uses cold plasma ozone generation to generate the ozone-air mixture.

31. The system of claim 21, wherein the indoor area comprises a plurality of indoor areas, and wherein the ozone-air mixture is provided to one of the plurality of indoor areas at a time.

32. The system of claim 21, wherein the indoor area comprises a plurality of indoor areas, and wherein the ozone-air mixture is provided simultaneously to at least two of the plurality of indoor areas.

33. A system for controlling a provision of an ozone for disinfecting an indoor area, the system comprising:
an ozone generator for generating the ozone, wherein the ozone generator comprising a temperature sensor and a high-voltage sensor;
a motion sensor connected to the ozone generator and configured for sensing a presence of a human in the indoor area;
an ozone concentration sensor connected to the ozone generator and configured for sensing an ozone concentration in the indoor area;
a distribution module configured for selectively distributing the ozone from the ozone generator to the indoor area according to a distribution control signal received from the ozone generator;
a pressure sensor connected to the ozone generator and configured for sensing a pressure of the ozone; and
a plurality of control modules connected to the ozone generator and configured to enter one of a normal operation mode, an emergency shutdown mode, and an emergency lockdown mode.

34. The system of claim 33, wherein the normal operating mode comprises executing a schedule configured for commanding the ozone generator to receive, process according to the schedule, and transmit to at least one of the plurality of control modules a set of signals from the temperature sensor, the high-voltage sensor, the motion sensor, and the ozone concentration sensor, and for commanding the distribution module to selectively distribute ozone to the indoor area according to the schedule and the set of signals from the temperature sensor, the high-voltage sensor, the motion sensor, and the ozone concentration sensor after receiving a signal from one of the plurality of control modules.

35. The system of claim 33, wherein the emergency shutdown mode is entered upon the ozone generator detecting one of a loss of connection between one of the plurality of control modules and the ozone generator, and a signal sourced from one of the ozone concentration sensor exceeding a first threshold, the temperature sensor exceeding a second threshold, the pressure sensor substantially differing from a third threshold, or one of the plurality of control modules detecting a problem with a predetermined component.

36. The system of claim 33, wherein the emergency lockdown mode is entered upon the ozone generator detecting one of a loss of connection between one of the plurality of control modules and the ozone generator, and a signal sourced from one of the ozone concentration sensor exceeding a first threshold for a first time period, the temperature sensor exceeding a second threshold for a second time period, the pressure sensor substantially differing from a third threshold for a third time period, one of the plurality of control modules detecting a problem with a predetermined component for a fourth time period, or the high-voltage sensor dropping below a fourth threshold, wherein the one of the plurality of control modules is physically remote to the ozone generator.

37. A non-transitory computer program product including a computer readable medium for controlling a provision of an ozone for disinfecting an indoor area, wherein the computer readable medium having a set of instructions to execute a schedule at a main control module under a normal operation mode, wherein the set of instructions is configured for:
commanding an ozone generator to receive, process according to the schedule, and transmit to a local control module and to the main control module, a set of signals from a temperature sensor installed in an ozone generation chamber of an ozone generator, a high-voltage sensor connected to the ozone generator, a motion sensor installed at the indoor area, and an ozone concentration sensor installed at the indoor area;
commanding a compressor to provide an air to the ozone generator; and
commanding a distribution module to selectively distribute an ozone-air mixture to the indoor area according to the schedule and the set of signals from the temperature sensor, the high-voltage sensor, the motion sensor, and the ozone concentration sensor after receiving at the local control module a signal from the main control module;
wherein the ozone generator is configured to enter one of an emergency shutdown mode and an emergency lockdown.

38. A method of controlling a provision of an ozone for disinfecting an indoor area by executing a schedule at a main control module under a normal operation mode, the method comprising:
commanding an ozone generator to receive, process according to the schedule, and transmit to a local control module and to the main control module, a set of signals from a temperature sensor installed in an ozone generation chamber of an ozone generator, a high-voltage sensor connected to the ozone generator, a motion sensor installed at the indoor area, and an ozone concentration sensor installed at the indoor area;
commanding a compressor to provide an air to the ozone generator; and
commanding a distribution module to selectively distribute an ozone-air mixture to the indoor area according to the schedule and the set of signals from the temperature sensor, the high-voltage sensor, the motion sensor, and the ozone concentration sensor after receiving at the local control module a signal from the main control module;
wherein the ozone generator is configured to enter one of an emergency shutdown mode and an emergency lockdown.

39. The method of claim 38, wherein the emergency shutdown mode is entered upon the ozone generator detecting one of a loss of connection between the main control module and the ozone generator, and a signal sourced from one of the ozone concentration sensor exceeding a first threshold, the temperature sensor exceeding a second threshold, the pressure sensor substantially differing from a third threshold, or the main control module detecting a problem with a predetermined component.

40. The method of claim 38, wherein the emergency lockdown mode is entered upon the ozone generator detecting one of a loss of connection between the main control module and the ozone generator, and a signal sourced from one of the ozone concentration sensor exceeding a first threshold for a first time period, the temperature sensor exceeding a second threshold for a second time period, the pressure sensor substantially differing from a third threshold for a third time period, the main control module detecting a problem with a predetermined component for a fourth time period, or the high-voltage sensor dropping below a fourth threshold.

41. A system comprising:
a generator configured to generate an ozone;
a device configured to output the ozone;
a first sensor configured to sense the ozone;
a second sensor configured to sense a human;
a first logic configured to communicate with the generator or the device when (i) the generator generates the ozone, (ii) the device outputs the ozone into a defined area while the first sensor sensing for the ozone within the defined area and the second sensor sensing for the human within the defined area, and (iii) the first logic is remote from the generator, the device, the first sensor, and the second sensor; and
a second logic configured to communicate with the generator or the device when (i) the generator generates the ozone, (ii) the device outputs the ozone into the defined area while the first sensor sensing for the ozone within the defined area and the second sensor sensing for the human within the defined area, (iii) the second logic is local to the generator, the device, the first sensor, and the second sensor, and (iv) the first logic is not in communication with the second logic, the generator, or the device.

42. A system comprising:
a first sensor configured to sense an ozone within a defined area;
a second sensor configured to sense a human within the defined area;
a generator configured to generate the ozone to be output into the defined area based on the first sensor and the second sensor;
a first logic remote from the generator, the first sensor, and the second sensor, wherein the first logic is configured to communicate with the generator; and
a second logic local to the generator, the first sensor, and the second sensor, wherein the second logic is configured to communicate with the generator.

43. A system comprising:
a generator configured to generate an ozone to be output into a defined area;
a first sensor configured to communicate with the generator and sense the ozone within the defined area;
a second sensor configured to communicate with the generator and sense a human within the defined area;
a first logic configured to communicate with the generator when (i) the generator generates the ozone to be output into a defined area while the first sensor sensing for the ozone within the defined area and the second sensor sensing for the human within the defined area and (ii) the first logic is remote from the generator, the first sensor, and the second sensor; and
a second logic configured to communicate with the generator when (i) the generator generates the ozone to be output into the defined area while the first sensor sensing for the ozone within the defined area and the second sensor sensing for the human within the defined area, (ii) the second logic is local to the generator, the first sensor, and the second sensor, and (iii) the first logic is not in communication with the second logic or the generator.

44. A system comprising:
a first sensor configured to sense an ozone within a defined area;
a second sensor configured to sense a human within the defined area;
a generator configured to generate the ozone to be output into the defined area based on the first sensor and the second sensor;
a first logic remote from the generator, the first sensor, and the second sensor; and
a second logic local to the generator, the first sensor, and the second sensor.
